(19) United States
(12) Patent Application Publication
(10) Pub. No.: US 2016/0361243 A1
(43) Pub. Date: Dec. 15, 2016

(54) Use Of Glucamides To Improve Silicone Deposition

(71) Applicant: Clariant International Ltd., Muttenz (CH)

(72) Inventors: Peter KLUG, Grossostheim (DE); Carina MILDNER, Geisenheim (DE)

(73) Assignee: Clariant International Ltd., Muttenz (CH)

(21) Appl. No.: 15/039,970

(22) PCT Filed: Nov. 27, 2014
(86) PCT No.: PCT/EP2014/003176

§ 371 Date: May 27, 2016

(30) Foreign Application Priority Data
Nov. 29, 2013 (DE) 102013018001.5

(51) Int. Cl. A61K 8/42; A61K 8/46; A61K 8/73; A61Q 5/12; A61K 8/49; A61K 8/19; A61Q 5/02; A61K 8/44; A61K 8/892

(52) U.S. Cl. CPC A61K 8/42; A61K 8/442; A61K 8/463; A61K 8/737; A61K 8/892; A61K 8/466; A61K 8/4946; A61K 8/19; A61Q 5/02; A61Q 5/12; A61K 2800/594; A61K 2800/596; A61K 2800/5426

(57) ABSTRACT

The invention relates to compositions, containing: at least one N-alkyl-N-acylglucamine as component A, dimethiconol as component B, at least one anionic surfactant as component C, optionally at least one betaine surfactant as component D, optionally at least one further surfactant as component E, water as component F, and optionally at least one further additive as component G, which compositions can be used as hair-washing agents.

The invention relates to compositions comprising N-alkyl-N-acylglucamines, dimethiconol and anionic surfactants, to the use thereof in a method of caring for the hair, and to the use of the composition for improving the silicone deposition. Furthermore, the invention relates to a method for producing a composition according to the invention.

Cosmetic compositions comprising fatty acid N-alkylpolyhydroxyalkylamides and fatty alcohols for caring for the skin and hair are known.

WO 97/47284 proposes cosmetic preparations for use in the hair care and skin care sector, comprising (a) ester quats and (b1) sorbitan esters, (b2) polyol poly-12-hydroxystearates and/or (b3) glycerides, and optionally (c1) alkyl and/or alkenyl oligoglycosides and/or (c2) fatty acid N-alkylpolyhydroxyalkylamides. The compositions are characterized by an improved soft feel of the hair and a particularly pleasant skin feel. Examples with fatty acid N-alkylpolyhydroxyalkylamides derived from coconut fatty acid are specifically disclosed.

WO 94/21226 discloses detergent mixtures comprising polyhydroxy fatty acid amides, monomeric cationic surfactants, fatty alcohols and optionally oil bodies, hair treatment products which comprise these mixtures, and the use of the mixtures for producing hair treatment products. Examples with C12/14 fatty acid N-alkylpolyhydroxyalkylamides are specifically disclosed.

Finely divided, storage-stable emulsions comprising, inter alia, fatty acid N-alkylpolyhydroxyalkylamides and fatty alcohols are known, for example, from WO 97/06870. Preference is given here to fatty acid N-alkylpolyhydroxyalkylamides based on lauric acid or C12/14 coconut fatty acid.

WO 96/27366 discloses cosmetic and pharmaceutical compositions which comprise, as O/W emulsifiers, fatty acid N-alkylglucamides in combination with fatty alcohols as co-emulsifiers. There is no reference to conditioning effect in hair care.

WO 92/05764 describes hair washing products which comprise, inter alia, glucamides and dimethicone.

Although good results have already been attained with the known compositions, there is still a great deal of room for improvements, particularly with regard to use in hair washing products.

It was an object, for example, to develop compositions which can be readily formulated and permit increased silicone deposition, especially when used as hair washing products.

It has been found that this object is achieved by compositions which comprise at least one N-alkyl-N-acylglucamine, dimethiconol and at least one anionic surfactant.

The invention therefore provides a composition comprising:
at least one N-alkyl-N-acylglucamine as component A,
dimethiconol as component B,
at least one anionic surfactant as component C,
optionally at least one betaine surfactant as component D,
optionally at least one further surfactant as component E,
water as component F,
optionally at least one further additive as component G.

When used for example as hair washing products, compositions according to the invention lead to excellent hair conditioning and to a considerably increased silicone deposition even if the amount of silicone used is small. Consequently, the amount of silicones used can be considerably reduced.

Preferably, compositions according to the invention are free from amphoteric surfactants and in particular free from betaines. A composition according to the invention particularly preferably comprises no cocamidopropyl betaine.

In the case of hair care products, compositions according to the invention have improved sensory properties and a very good hair conditioning effect. Moreover, they are biodegradable.

The N-alkyl-N-acylglucamines used according to the invention in which glucamine is preferably an N-1-deoxysorbityl group are particularly preferably N-alkyl-N-acylglucamines of the formula (I),

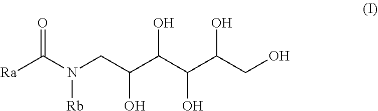

where, in the formula (I), RₐCO is a linear or branched, saturated or unsaturated C₆-C₂₂-acyl radical and R_{b} is a C₁-C₄ alkyl radical. Particularly preferably, R_{b} in formula (I) is a methyl radical (—CH₃) and RₐCO has the above meaning.

As component A, preference is given to saturated N-alkyl-N-acylglucamines of the formula (I), where the acyl radical RₐCO is derived from myristic acid, lauric acid, palmitic acid, stearic acid, oleic acid, linoleic acid or linolenic acid.

Preference is also given to N-alkyl-N-acylglucamines of the formula (I) in which RₐCO is derived from coconut oil or palm kernel oil, particularly preferably from coconut oil.

Coconut oil typically comprises triglycerides which comprise saturated fatty acid radicals which are derived from caprylic acid, lauric acid, capric acid, oleic acid, palmitic acid, stearic acid and myristic acid.

Coconut oil here comprises preferably

a) 40-55% by weight of lauric acid,
b) 10-20% by weight of myristic acid,
c) 8-12% by weight of palmitic acid,
d) 6-12% by weight of oleic acid and
h) 0-36% by weight of further fatty acids,
where the sum of the fatty acids bonded to the triglyceride is 100% by weight.

Particularly preferably, coconut oil comprises

a) 40-55% by weight of lauric acid,
b) 10-20% by weight of myristic acid,
c) 8-12% by weight of palmitic acid,
d) 6-12% by weight of oleic acid,
e) 5-10% by weight of decanoic acid,
f) 4-10% by weight of octanoic acid,
g) 1-3% by weight of stearic acid and
h) 0-26% by weight of further fatty acids,
where the sum of the fatty acids bonded to the triglyceride is 100% by weight.

Preferred N-alkyl-N-acylglucamines are compounds of the formula (I) in which RₐCO is a C₁₂-C₁₄-acyl radical. Particular preference is given to N-alkyl-N-acylglucamines of the formula (I) in which RₐCO is a C₁₂-C₁₄-acyl radical and R_{b} is a methyl radical.

In general, the term C₁₂-C₁₄-acyl radical can be understood as meaning a C₁₂-acyl radical or C₁₄-acyl radical or a mixture of the two.

Besides the N-alkyl-N-acylglucamines of the formula (I) in which RₐCO is a C₁₂-C₁₄-acyl radical, the compositions can have small fractions of N-alkyl-N-acylglucamines derived from short-chain and/or long-chain fatty acids, in particular those which comprise C₁-C₄-acyl, C₆-, C₈-, C₁₀-, C₁₆-, C₁₈- and/or C₂₀-acyl.

In a further embodiment, the N-alkyl-N-acylglucamine of a composition according to the invention is a mixture of at least one N-alkyl-N-acylglucamine of the formula (I) where RₐCO is a C₁₂-acyl radical, and at least one N-alkyl-N-acylglucamine of the formula (I) where RₐCO is a C₁₄-acyl radical.

Particularly preferably, the weight ratio of N-alkyl-N-acylglucamine of the formula (I) where RₐCO is a C₁₂-alkyl radical to N-alkyl-N-acylglucamine of the formula (I) where RₐCO is a C₁₄-alkyl radical is 50:50 to 90:10, in particular 60:40 to 80:20.

Preferably, in such mixtures of component A, the content of N-alkyl-N-acylglucamines of the formula (I) in which RₐCO is a C₁₂-C₁₄-acyl radical is 70% by weight, preferably 90% by weight, based on the content of component A.

Preferably, the fraction of component A in a composition according to the invention is 0.1-5.0% by weight, based on the composition, and particularly preferably 1.0-3.0% by weight, based on the composition.

The N-alkyl-N-acylglucamines used here can be prepared, as described in EP 0 550 637 A1, by reacting the corresponding fatty acid esters or fatty acid ester mixtures with N-alkyl glucamine in the presence of a solvent having hydroxyl groups or alkoxyl groups. Suitable solvents are, for example, C₁-C₄-monoalcohols, ethylene glycol, propylene glycol, glycerol, and alkoxylated alcohols. Preference is given to 1,2-propylene glycol. N-alkyl glucamine can be obtained, as likewise described in EP 0 550 637 A1, by reductive amination of glucose with alkylamine.

Suitable fatty acid esters which are reacted with the N-alkyl glucamines to give N-alkyl-N-acylglucamines are generally the alkyl esters, specifically the methyl or ethyl esters, which are obtained by transesterification from natural fats and oils, for example the triglycerides, or triglycerides, such as e.g. coconut oil.

Suitable raw materials for the preparation of the fatty acid alkyl esters are, for example, coconut oil or palm oil, with coconut oil being particularly preferred.

The compositions according to the invention comprise dimethiconol as component B (formula II).

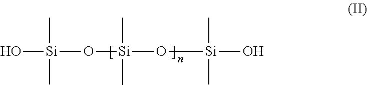

In formula (II), “n” is preferably 10-10 000, particularly preferably 100-5000.

Since high molecular weight dimethiconols are very highly viscous substances, they are generally supplied as solutions in low molecular weight silicones such as cyclomethicone or dimethicone or as a dispersion in water.

Based on the total composition, the content of component B can generally be from 0.1 to 5.0% by weight. Preferably, the content of component B, based on the total composition, is from 0.3 to 2.0% by weight.

Preferably, the weight ratio between N-alkyl-N-acylglucamine:dimethiconol is from 10:1 to 1:1, where the ratio between N-alkyl-N-acylglucamine:dimethiconol is particularly preferably from 5:1 to 1.5:1.

The composition according to the invention furthermore comprises at least one anionic surfactant as component C, preferably from the group of alkyl sulfates and alkyl ether sulfates.

Preferred alkyl sulfates are the C₈-C₂₀-alkyl sulfates, in particular the linear C₈-C₂₀-alkyl sulfates preferably in the form of their sodium, potassium or ammonium salts. Examples of alkyl sulfates are lauryl sulfate, coco alkyl sulfate and tallow alkyl sulfate. Particular preference is given to lauryl sulfate.

Preferred alkyl ether sulfates are the C₈-C₂₀-alkyl ether sulfates, particularly preferably the linear C₈-C₂₀-alkyl ether sulfates, in particular the alkyl glycol ether sulfates derived from the ethoxylated fatty alcohols, in the form of their sodium, potassium or ammonium salts. Examples of alkyl ether sulfates are lauryl ether sulfate, myristyl ether sulfate, coco alkyl ether sulfate and tallow alkyl ether sulfate. Particular preference is given to lauryl ether sulfate. Examples of glycol ether sulfates are lauryl triethylene glycol ether sulfate, coco alkyl triethylene glycol ether sulfate and tallow alkyl hexaethylene glycol ether sulfate. Particular preference is given to lauryl glycol ether sulfate, for example lauryl diethylene glycol ether sulfate or lauryl triethylene glycol ether sulfate, specifically in the form of the sodium salts.

A particularly preferred anionic surfactant is sodium lauryl ether sulfate.

In a further embodiment of the invention, the compositions comprise one or more N-acyl amino acid surfactants as anionic surfactants. In the context of a preferred embodiment, the amino acid radical of such N-acyl amino acid surfactants is selected from the group consisting of proteinogenic amino acids, the N-alkylated derivatives thereof or mixtures thereof.

Particularly preferred N-acyl amino acid surfactants are acyl glycinates, acyl alaninates, acyl aspartates, acyl glutamates, acyl sarcosinates or mixtures thereof. The N-acyl amino acid surfactants are very particularly preferably selected from the group consisting of acyl glycinate, acyl aspartate, acyl glutamate, acyl sarcosinate and mixtures thereof.

Very particularly preferably, the N-acylamino acid surfactants consist of at least one C₈-C₂₂-acylated amino acid, in particular N-alkylated derivatives thereof. Preference is given to the corresponding lauroyl or cocoyl derivatives of the amino acids.

Particular preference is therefore given to sodium cocoyl glycinate, potassium cocoyl glycinate, sodium lauroyl glycinate, potassium lauroyl glycinate, sodium cocoyl glutamate, sodium lauroyl glutamate, sodium cocoyl aspartate, sodium lauroyl aspartate and sodium lauroyl sarcosinate.

Preferably, the fraction of component C in the composition according to the invention is 5.0% by weight to 20.0% by weight, based on the composition, particularly preferably 10.0% by weight to 17.0% by weight, based on the composition.

In one embodiment, the compositions according to the invention comprise a betaine surfactant D as well as the at least one anionic surfactant.

Betaine surfactants D comprise, in the same molecule, a cationic group, in particular an ammonium group, and an anionic group, which can be a carboxylate group, sulfate group or sulfonate group. Suitable betaines are alkyl betaines such as cocobetaine or fatty acid alkyl amidopropyl betaines, for example cocoacylamidopropyldimethyl betaine, C₁₂-C₁₈-dimethyl aminohexanoates or C₁₀-C₁₈-acyl amidopropane dimethyl betaines.

In a preferred embodiment of the invention, the compositions comprise one or more amidopropyl betaines of the general formula (III),

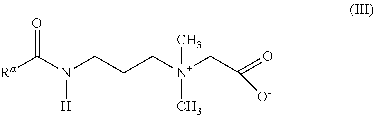

in which R^{a} is a linear or branched saturated C₇-C₂₁ alkyl group or a linear or branched mono- or polyunsaturated C₇-C₂₁ alkenyl group.

In a further embodiment of the invention, the compositions comprise one or more betaines of the formula (IV),

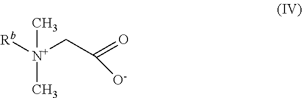

in which R^{b} is a linear or branched saturated C₈-C₂₂ alkyl group or a linear or branched mono- or polyunsaturated C₈-C₂₂ alkenyl group.

In a further embodiment of the invention, the compositions comprise one or more sulfobetaines of the formula (V),

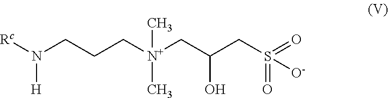

in which R^{c} is a linear or branched saturated C₈-C₂₂ alkyl group or a linear or branched mono- or polyunsaturated C₈-C₂₂ alkenyl group.

Preferably, the compositions comprise, besides one or more alkyl sulfates and/or alkyl ether sulfates, one or more betaine surfactants selected from the group of the compounds consisting of the amidopropyl betaines of the formula (III), the betaines of the formula (IV) and the sulfobetaines of the formula (V).

In a particularly preferred embodiment of the invention, the compositions comprise one or more betaine surfactants selected from the amidopropyl betaines of the formula (III).

In a further particularly preferred embodiment of the invention, the compositions comprise one or more betaine surfactants selected from the betaines of the formula (IV).

In a further particularly preferred embodiment of the invention, the compositions comprise one or more betaine surfactants selected from the sulfobetaines of the formula (V).

Preferably, the radical R^{a} in the one or more amidopropyl betaines of the formula (III) is a linear or branched saturated C₇-C₁₇ alkyl group. Among the linear and branched saturated alkyl groups R^{a}, the linear saturated alkyl groups are preferred.

The amidopropyl betaines of the formula (III) are particularly preferably cocamidopropyl betaines. Preferred betaine surfactants are thus cocamidopropyl betaine, and alkyl betaines such as cocobetaine.

Preferably, the radical R^{b} in the one or more betaines of the formula (III) is a linear or branched saturated C₈-C₁₈-alkyl group and particularly preferably a linear or branched saturated C₁₂-C₁₈-alkyl group. Among the linear and branched saturated alkyl groups R^{b}, the linear saturated alkyl groups are preferred.

Preferably, the radical R^{c} in the one or more sulfobetaines of the formula (V) is a linear or branched saturated C₈-C₁₈ alkyl group and particularly preferably a linear or branched saturated C₁₂-C₁₈ alkyl group. Among the linear and branched saturated alkyl groups R^{c}, the linear saturated alkyl groups are preferred.

As (optionally used) further surfactants, cationic, nonionic and/or amphoteric surfactants can be used.

Suitable cationic surfactants are substituted or unsubstituted straight-chain or branched quaternary ammonium salts of the type R¹N(CH₃)₃X, R¹R²N(CH₃)₂X, R¹R²R³N(CH₃)X or R¹R²R³R⁴NX. The radicals R¹, R², R³ and R⁴ can preferably be, independently of one another, unsubstituted alkyl with a chain length between 8 and 24 carbon atoms, in particular between 10 and 18 carbon atoms, hydroxyalkyl having 1 to 4 carbon atoms, phenyl, C₂- to C₁₈-alkenyl, C₇- to C₂₄-aralkyl, (C₂H₄O)ₓH, where x is from 1 to 3, one or more alkyl radicals containing ester groups, or cyclic quaternary ammonium salts. X is a suitable anion. Preference is given to (C₈-C₂₂)-alkyltrimethylammonium chloride or bromide, particularly preferably cetyltrimethylammonium chloride or bromide, di(C₈-C₂₂)-alkyldimethylammonium chloride or bromide, (C₈-C₂₂)-alkyldimethylbenzylammonium chloride or bromide, (C₈-C₂₂)-alkyldimethylhydroxyethylammonium chloride, phosphate, sulfate, lactate, particularly preferably distearyldimethylammonium chloride, di(C₈-C₂₂)-alkylamidopropyltrimethylammonium chloride and methosulfate.

The amount of cationic surfactants in the compositions according to the invention can be up to 5% by weight, based on the total weight of the finished compositions.

Suitable nonionic surfactants are, for example, the following compounds:

polyethylene, polypropylene and polybutylene oxide condensates of alkylphenols. These compounds comprise the condensation products of alkylphenols with a C₆- to C₂₀-alkyl group, which can either be linear or branched, with alkene oxides. The surfactants are referred to as alkylphenol alkoxylates, e.g. alkylphenol ethoxylates.

Condensation products of aliphatic alcohols having 1 to 25 mol of ethylene oxide. The alkyl or alkenyl chain of the aliphatic alcohols can be linear or branched, primary or secondary, and generally contains 8 to 22 carbon atoms. The condensation products of C₁₀- to C₂₀-alcohols with 2 to 18 mol of ethylene oxide per mol of alcohol are particularly preferred. The alcohol ethoxylates can have a narrow homolog distribution of the ethylene oxide (“narrow range ethoxylates”) or a broad homolog distribution of the ethylene oxide (“broad range ethoxylates”). Examples of commercially available nonionic surfactants of this type are Tergitol® 15-S-9 (condensation product of a linear secondary C₁₁-C₁₅-alcohol with 9 mol of ethylene oxide), Tergitol® 24-L-NMW (condensation product of a linear primary C₁₂-C₁₄-alcohol with 6 mol of ethylene oxide coupled with a narrow molecular weight distribution). This product class likewise includes the Genapol® brands from Clariant.

Condensation products of ethylene oxide with a hydrophobic base, formed by condensation of propylene oxide with propylene glycol. The hydrophobic moiety of these compounds preferably has a molecular weight between 1500 and 1800. The addition of ethylene oxide onto this hydrophobic moiety leads to an improvement in solubility in water. The product is liquid up to a polyoxyethylene content of approx. 50% of the total weight of the condensation product, which corresponds to a condensation with up to approx. 40 mol of ethylene oxide. Commercially available examples of this product class are the Pluronic® brands from BASF and the Genapol® PF brands from Clariant.

Condensation products of ethylene oxide with a reaction product of propylene oxide and ethylene diamine. The hydrophobic unit of these compounds consists of the reaction product of ethylene diamine with excess propylene oxide and generally has a molecular weight from 2500 to 3000. Ethylene oxide is added onto this hydrophobic unit up to a content of 40 to 80% by weight of polyoxyethylene and a molecular weight of from 5000 to 11 000. Commercially available examples of this compound class are the Tetronic® brands from BASF and the Genapol® PN brands from Clariant.

Preferred nonionic surfactants are fatty alcohol ethoxylates (alkyl polyethylene glycols); alkylphenol polyethylene glycols; fatty amine ethoxylates (alkylamino polyethylene glycols); fatty acid ethoxylates (acyl polyethylene glycols); polypropylene glycol ethoxylates (Pluronics®); fatty acid alkanolamides, (fatty acid amide polyethylene glycols); sucrose esters, sorbitol esters and sorbitan esters and their polyglycol ethers, and C₈-C₂₂-alkyl polyglucosides.

The amount of nonionic surfactants in the compositions according to the invention (e.g. in the case of rinse-off products) is preferably in the range from 0.1 to 10.0% by weight, particularly preferably from 0.5 to 5.0% by weight and especially preferably from 1.0 to 3.0% by weight.

Furthermore, the compositions according to the invention can comprise amphoteric surfactants which do not belong to the group of betaines of component (D). These can be described as derivatives of long-chain secondary or tertiary amines, which have an alkyl group having 8 to 18 carbon atoms and in which a further group is substituted with an anionic group which imparts the solubility in water, thus e.g. with a carboxyl, sulfate or sulfonate group. Preferred amphoteric surfactants are N—(C₁₂-C₁₈)-alkyl-3-aminopropionates and N—(C₁₂-C₁₈)-alkyl-β-iminodipropionates as alkali metal and mono-, di- and trialkylammonium salts. Suitable further surfactants are also amine oxides. These are oxides of tertiary amines with a long-chain group of 8 to 18 carbon atoms and two mostly short-chain alkyl groups having 1 to 4 carbon atoms. Preference is given here, for example, to the C₁₀- to C₁₈-alkyldimethylamine oxides and fatty acid amidoalkyldimethylamine oxides.

The amount of the amphoteric surfactants is preferably from 0.5 to 20.0% by weight and particularly preferably from 1.0 to 10.0% by weight, based on the composition.

In a preferred embodiment, the compositions according to the invention also additionally comprise cosurfactants from the group of fatty acid alkanolamides as foam-boosting agents.

The compositions according to the invention comprise water as component F.

Preferably, the content of water is 60 to 90% by weight, based on the composition, in particular 70 to 85% by weight, based on the composition.

The compositions according to the invention can have, as one or more additives G, preservatives, fragrances, dyes, cationic polymers, thickeners and gelling agents, pigments, antimicrobial and biogenic active ingredients, moisturizing agents, stabilizers, acids and/or alkalies.

Suitable preservatives are the preservatives listed in the relevant annex of the European Cosmetic Legislation, for example phenoxyethanol, benzyl alcohol, parabens, benzoic acid and sorbic acid, of particularly good suitability is, for example, 1,3-bis(hydroxymethyl)-5,5-dimethylimidazolidine-2,4-dione (Nipaguard® DMDMH).

Fragrances that can be used are scented oils or perfume oils. Scented oils or perfume oils can be individual fragrance compounds, e.g. the synthetic products of the ester, ether, aldehyde, ketone, alcohol and hydrocarbon type. Fragrance compounds of the ester type are e.g. benzyl acetate, phenoxyethyl isobutyrate, p-tert-butyl cyclohexyl acetate, linalyl acetate, dimethylbenzylcarbinyl acetate, phenylethyl acetate, linalyl benzoate, benzyl formate, ethyl methylphenyl glycinate, allyl cyclohexyl propionate, styrallyl propionate and benzyl salicylate. The ethers include, for example, benzyl ethyl ether, the aldehydes include e.g. the linear alkanals having 8 to 18 carbon atoms, citral, citronellal, citronellyloxy acetaldehyde, cyclamenaldehyde, hydroxycitronellal, lilial and bourgeonal, and the ketones include e.g. the ionones, alpha-isomethylionone and methyl cedryl ketone, the alcohols include anethole, citronellol, eugenol, geraniol, linalol, phenylethyl alcohol and terpineol, the hydrocarbons include primarily the terpenes and balsams. Preference is given to using mixtures of different fragrances which together produce a pleasant scent note.

Perfume oils can also comprise natural fragrance mixtures, as are accessible from vegetable or animal sources, e.g. pine oil, citrus oil, jasmine oil, lily oil, rose oil or ylang ylang oil. Essential oils of relatively low volatility, which are mostly used as aroma components, are also suitable as perfume oils, e.g. sage oil, camomile oil, clove oil, melissa oil, mint oil, cinnamon leaf oil, linden blossom oil, juniper berry oil, vetiver oil, olibanum oil, galbanum oil and ladanum oil.

The dyes and pigments present in the compositions according to the invention, both organic and inorganic dyes, can be selected from the corresponding positive list of the Cosmetics Directive or from the EU list of cosmetic colorants. Pearlescent pigments are also advantageously used, e.g. pearl essence (guanine/hypoxanthine mixed crystals from fish scales) and mother of pearl (ground mussel shells), monocrystalline pearlescent pigments such as e.g. bismuth oxychloride (BiOCl), layer-substrate pigments, e.g. mica/metal oxide, silver-white pearlescent pigments of TiO₂, interference pigments (TiO₂, varying layer thickness), color luster pigments (Fe₂O₃) and combination pigments (TiO₂/Fe₂O₃, TiO₂/Cr₂O₃, TiO₂/Prussian blue, TiO₂/carmine).

The amount of dyes and pigments in the compositions according to the invention is generally from 0.01 to 1.0% by weight, based on the total weight of the finished compositions.

Suitable cationic polymers are those known under the INCI name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and also Polyquaternium 37 & mineral oil & PPG trideceth (Salcare SC95), PVP-dimethylaminoethyl methacrylate copolymer, guar hydroxypropyltriammonium chlorides, and calcium alginate and ammonium alginate. It is furthermore possible to use cationic cellulose derivatives; cationic starch; copolymers of diallylammonium salts and acrylamides; quaternized vinylpyrrolidonelvinylimidazole polymers; condensation products of polyglycols and amines; quaternized collagen polypeptides; quaternized wheat polypeptides; polyethyleneimines; cationic silicone polymers, such as e.g. amidomethicones; copolymers of adipic acid and dimethylaminohydroxypropyldiethylenetriamine; polyamino polyamide and cationic chitin derivatives, such as, for example, chitosan.

In a further preferred embodiment, a composition according to the invention has a galactomannan compound as cationic polymer. Preference here is given to galactomannan 2-hydroxypropyltrimethylammonium chloride ether.

The compositions according to the invention can comprise one or more of the aforementioned cationic polymers in amounts of from 0.1 to 5.0% by weight, preferably from 0.2 to 3.0% by weight and particularly preferably from 0.5 to 2.0% by weight, based on the finished compositions.

The desired viscosity of the compositions can be established by adding thickeners and gelling agents. Suitable substances are preferably cellulose ethers and other cellulose derivatives (e.g. carboxymethylcellulose, hydroxyethylcellulose), gelatin, starch and starch derivatives, sodium alginates, fatty acid polyethyleneglycol esters, fatty acid amides, fatty acid alkanolamides; alcohol-soluble polyamides and polyacrylamides or mixtures of such. Furthermore, crosslinked and uncrosslinked polyacrylates such as carbomers, sodium polyacrylates or polymers containing sulfonic acid, such as ammonium acryloyldimethyltaurate/carboxyethyl acrylate crosspolymer, can be used.

Preferably, the compositions according to the invention comprise from 0.01 to 10.0% by weight, particularly preferably from 0.1 to 5.0% by weight, especially preferably from 0.2 to 3.0% by weight and very particularly preferably from 0.4 to 2.0% by weight, of thickeners or gelling agent.

The antimicrobial active ingredients used are for example, cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoyl glycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctoses, in particular zinc salts, pyrithiones and heavy metal salts thereof, especially zinc pyrithione, zinc phenolsulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and octopirox, iodopropynyl butylcarbamate, methylchloroisothiazolinone, methylisothiazolinone, methyldibromoglutaronitrile, AgCl, chloroxylenol, Na salt of diethylhexyl sulfosuccinate, sodium benzoate, and phenoxyethanol, benzyl alcohol, phenoxyisopropanol, parabens, preferably butyl, ethyl, methyl and propyl paraben, and Na salts thereof, pentanediol, 1,2-octanediol, 2-bromo-2-nitropropane-1,3-diol, ethylhexylglycerol, benzyl alcohol, sorbic acid, benzoic acid, lactic acid, imidazolidinylurea, diazolidinylurea, dimethyloldimethylhydantoin (DMDMH), Na salt of hydroxymethylglycinate, hydroxyethylglycine of sorbic acid and combinations of these active substances.

The compositions according to the invention comprise the antimicrobial active ingredients preferably in amounts of from 0.001 to 5.0% by weight, particularly preferably from 0.01 to 3.0% by weight and especially preferably from 0.1 to 2.0% by weight, based on the finished compositions.

The compositions according to the invention can furthermore comprise biogenic active ingredients selected from plant extracts such as, for example, aloe vera, and also local anesthetics, antibiotics, antiphlogistics, antiallergics, corticosteroids, sebostatics, Bisabolol® Allantoin®, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, and also vitamin E and/or derivatives thereof.

The compositions according to the invention can comprise biogenic active ingredients preferably in amounts of from 0.001 to 5.0% by weight, particularly preferably from 0.01 to 3.0% by weight and especially preferably from 0.1 to 2.0% by weight, based on the finished compositions.

The moisturizing substance is, for example, isopropyl palmitate, glycerol glycerylglucoside and/or sorbitol.

The compositions according to the invention are preferably adjusted to a 25 pH in the range from 2 to 12, preferably in the range from 3 to 9.

The acids or alkalies used for adjusting the pH are preferably mineral acids, in particular HCl, inorganic bases, in particular NaOH or KOH, or organic acids, in particular citric acid.

Furthermore, the compositions according to the invention can comprise oil bodies. The oil bodies can advantageously be selected from the groups of triglycerides, natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids or from the group of alkyl benzoates, as well as natural or synthetic hydrocarbon oils.

Of suitability are triglycerides of linear or branched, saturated or unsaturated, optionally hydroxylated, C₈-C₃₀-fatty acids, in particular vegetable oils, such as sunflower oil, corn oil, soya oil, rice oil, jojoba oil, babussu oil, pumpkin oil, grape seed oil, sesame oil, walnut oil, apricot oil, orange oil, wheat germ oil, peach kernel oil, macadamia oil, avocado oil, sweet almond oil, lady's smock oil, castor oil, olive oil, peanut oil, rapeseed oil and coconut oil, as well as synthetic triglyceride oils, e.g. the commercial product Myritol® 318. Hydrogenated triglycerides are also preferred in accordance with the invention. Oils of animal origin, for example bovine tallow, perhydrosqualene, lanolin, can also be used.

A further class of preferred oil bodies are the benzoic acid esters of linear or branched C₈₋₂₂-alkanols, e.g. the commercial products Finsolv® SB (isostearylbenzoate), Finsolv® TN (C₁₂-C₁₅-alkylbenzoate) and Finsolv® EB (ethylhexylbenzoate).

A further class of preferred oil bodies are the dialkyl ethers having in total 12 to 36 carbon atoms, in particular having 12 to 24 carbon atoms, such as e.g. di-n-octyl ether (Cetiol® OE), di-n-nonyl ether, di-n-decyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, di-3-ethyldecyl ether, tert-butyl n-octyl ether, isopentyl n-octyl ether and 2-methylpentyl n-octyl ether, and di-tert-butyl ether and diisopentyl ether.

Likewise of suitability are branched saturated or unsaturated fatty alcohols having 6-30 carbon atoms, e.g. isostearyl alcohol, and Guerbet alcohols.

A further class of preferred oil bodies is hydroxycarboxylic acid alkyl esters. Preferred hydroxycarboxylic acid alkyl esters are full esters of glycolic acid, lactic acid, malic acid, tartaric acid or citric acid. Further esters of hydroxycarboxylic acids that are suitable in principle are esters of β-hydroxypropionic acid, of tartronic acid, of D-gluconic acid, saccharic acid, mucic acid or glucuronic acid. Suitable alcohol components of these esters are primary, linear or branched aliphatic alcohols having 8 to 22 carbon atoms. Here, particular preference is given to the esters of C₁₂-C₁₅-fatty alcohols. Esters of this type are commercially available, e.g. under the trade name Cosmacol® from EniChem, Augusta Industriale.

A further class of preferred oil bodies is that of dicarboxylic acid esters of linear or branched C₂-C₁₀-alkanols, such as di-n-butyl adipate (Cetiol® B), di(2-ethylhexyl) adipate and di(2-ethylhexyl) succinate, and also diol esters such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate, and diisotridecyl azelate.

Likewise preferred oil bodies are symmetrical, asymmetrical or cyclic esters of carbonic acid with fatty alcohols, glycerol carbonate or dicaprylyl carbonate (Cetiol® CC).

A further class of preferred oil bodies are the esters of dimers of unsaturated C₁₂-C₂₂-fatty acids (dimer fatty acids) with monohydric linear, branched or cyclic C₂-C₁₈-alkanols or with highly hydric linear or branched C₂-C₆-alkanols.

A further class of preferred oil bodies is that of hydrocarbon oils, for example those with linear or branched, saturated or unsaturated C₇-C₄₀-carbon chains, for example Vaseline, dodecane, isododecane, cholesterol, lanolin, synthetic hydrocarbons such as polyolefins, in particular polyisobutene, hydrogenated polyisobutene, polydecane, and hexadecane, isohexadecane, paraffin oils, isoparaffin oils, e.g. the commercial products of the Permethyl® series, squalane, squalene, and alicyclic hydrocarbons, e.g. the commercial product 1,3-di(2-ethylhexyl)cyclohexane (Cetiol® S), ozokerite and ceresin.

A composition according to the invention can furthermore comprise additional further silicone oils or waxes different from dimethiconol as component B. Available silicone oils or waxes are preferably dimethylpolysiloxanes and cyclomethicones, polydialkylsiloxanes R₃—SiO(R₂SiO)ₓSiR₃, where R is methyl or ethyl, particularly preferably methyl, and x is a number from 2 to 500, for example the dimethicones available under the trade names VICASIL (General Electric Company), DOW CORNING 200, DOW CORNING 225, DOW CORNING 200 (Dow Corning Corporation), as well as the dimethicones available under SilCare® Silicone 41M65, SilCare® Silicone 41M70, SilCare® Silicone 41M80 (Clariant), stearyldimethylpolysiloxane, C₂₀-C₂₄-alkyldimethylpolysiloxane, C₂₄-C₂₈-alkyldimethylpolysiloxane, but also the methicones available under SilCare® Silicone 41M40, SilCare® Silicone 41M50 (Clariant), also trimethylsiloxysilicates [(CH₂)₃SiO)_{1/2}]ₓ[SiO₂]_{y}, where x is a number from 1 to 500 and y is a number from 1 to 500, R₃SiO[R₂SiO]ₓSiR₂OH, where R is methyl or ethyl and x is a number up to 500, HOR₂SiO[R₂SiO]ₓSiR₂OH, where R is ethyl and x is a number up to 500, polyalkylarylsiloxanes, for example the polymethylphenylsiloxanes available under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty-acid-, alcohol-, polyether-, epoxy-, fluorine- and/or alkyl-modified silicone compounds, and polyethersiloxane copolymers. In one embodiment, the composition comprises no further silicone oils and/or waxes different from dimethiconol.

Furthermore, the compositions according to the invention can comprise film formers which are selected, depending on the intended use, from salts of phenylbenzimidazole sulfonic acid, water-soluble polyurethanes, for example C₁₀-polycarbamyl polyglyceryl ester, polyvinyl alcohol, polyvinylpyrrolidone copolymers such as PVP/hexanedecene or PVP/eicosene copolymer, for example vinylpyrrolidone/vinyl acetate copolymer, water-soluble acrylic acid polymers/copolymers or esters or salts thereof, for example partial ester copolymers of acrylic/methacrylic acid and polyethylene glycol ethers of fatty alcohols, such as acrylate/steareth-20 methacrylate copolymer, water-soluble cellulose, for example hydroxymethyl cellulose, hydroxyethyl cellulose, hydroxypropyl cellulose, water-soluble quaterniums, polyquaterniums, carboxyvinyl polymers, such as carbomers and salts thereof, polysaccharides, for example polydextrose and glucan, vinyl acetate/crotonate, available for example under the trade name Aristoflex® A 60 (Clariant), and polymeric amine oxides, for example representatives available under the trade names Diaformer Z-711, 712, 731, 751.

The compositions according to the invention can comprise one or more film formers in amounts of from 0.1 to 10.0% by weight, preferably from 0.2 to 5.0% by weight and particularly preferably from 0.5 to 3.0% by weight, based on the finished compositions.

Furthermore, a composition according to the invention can also comprise superfatting agents. Superfatting agents that can be used are preferably lanolin and lecithin, non-ethoxylated and polyethoxylated or acylated lanolin and lecithin derivatives, polyol fatty acid esters, mono-, di- and triglycerides and/or fatty acid alkanolamides, where the latter simultaneously serve as foam stabilizers, which are used preferably in amounts of from 0.01 to 10.0% by weight, particularly preferably from 0.1 to 5.0% by weight and especially preferably from 0.5 to 3.0% by weight.

Compositions according to the invention can also have pearlescence-imparting components. Suitable pearlescence-imparting components are preferably fatty acid monoalkanolamides, fatty acid dialkanolamides, monoesters or diesters of alkylene glycols, in particular ethylene glycol and/or propylene glycol or oligomers thereof, with higher fatty acids, such as e.g. palmitic acid, stearic acid and behenic acid, monoesters or polyesters of glycerol with carboxylic acids, fatty acids and metal salts thereof, ketosulfones or mixtures of the specified compounds. Particular preference is given to ethylene glycol distearates and/or polyethylene glycol distearates with, on average, 3 glycol units.

If the compositions according to the invention comprise pearlescence-imparting compounds, these are present preferably in an amount of from 0.1 to 15.0% by weight, and particularly preferably in an amount of from 1.0 to 10.0% by weight, in the compositions according to the invention.

The total fraction of further additives as component G in the compositions according to the invention is preferably from 1.0 to 25.0% by weight and particularly preferably from 2.0 to 20.0% by weight, based on the composition.

In a preferred embodiment, a composition according to the invention comprises:
at least one N-alkyl-N-acylglucamine of the formula (I), where RₐCO is a C₁₂-acyl radical,
at least one N-alkyl-N-acylglucamine of the formula (I), where RₐCO is a C₁₄-acyl radical,
dimethiconol of the formula (V),
at least one lauryl ether sulfate, in particular sodium lauryl ether sulfate, as anionic surfactant,
no betaine surfactant, in particular no cocamidopropyl betaine.

In a further preferred embodiment, a composition according to the invention comprises:
a mixture of an N-alkyl-N-acylglucamine of the formula (I), where RₐCO is derived from coconut oil,
dimethiconol of the formula (V),
at least one lauryl ether sulfate, in particular sodium lauryl ether sulfate, as anionic surfactant,
no betaine surfactant, in particular no cocamidopropyl betaine.

Preferably, the compositions according to the invention comprise:
0.1-5.0% by weight, based on the composition, of component A,
0.1-5.0% by weight, based on the composition, of component B,
5.0-20.0% by weight, based on the composition, of component C,
0-10.0% by weight, based on the composition, of component D.

Particularly preferably, the compositions according to the invention comprise:
1.0-3.0% by weight, based on the composition, of component A,
1.0-3.0% by weight, based on the composition, of component B,
10.0-17.0% by weight, based on the composition, of component C,
1.0-5.0% by weight, based on the composition, of component D.

Particularly preferably, the composition according to the invention comprises no amphoteric surfactant, preferably no betaine and in particular no cocamidopropyl betaine.

The compositions according to the invention are preferably hair washing products for greasy hair, dry hair, stressed hair, flaky hair, a color shampoo, baby shampoo or sport shampoo.

Furthermore, the invention provides a method of treating and caring for the hair with a composition according to the invention. In the method according to the invention, the hair is brought into contact with a composition according to the invention. The composition here is preferably used as a “rinse-off” product.

Furthermore, the invention provides the use of the composition according to the invention for improving the silicone deposition.

Furthermore, the invention provides a method for producing a composition according to the invention. In the method according to the invention, the components A, B, C and F and optionally the components D, E and/or G are brought into contact with one another.

The examples below serve to illustrate the invention without, however, limiting it thereto.

### EXAMPLES

The N-alkyl-N-acylglucamines described below were prepared in accordance with EP 0 550 637 from the corresponding fatty acid methyl esters and N-acylglucamide in the presence of 1,2-propylene glycol as solvent and obtained as solid consisting of active substance, i.e. N-alkyl-N-acylglucamine, and 1,2-propylene glycol (all data in % by weight).

TABLE 1
Preparation examples for N-alkyl-N-acylglucamine

| | | | | 1,2- | |
|---|---|---|---|---|---|
| Prepa- | | | Active | propylene | Melting |
| ration | Methyl | | substance | glycol | point |
| example | ester | Triglyceride | (%) | (%) | (° C.) |
| 1 | C_{12/14} | — | 90 | 10 | 85 |
| | (C₁₂: 70%; | | | | |
| | C₁₄: 30%) | | | | |
| 2 | C_{16/18} | — | 80 | 20 | 65 |
| | (C₁₆: 60%; | | | | |
| | C₁₈: 40%) | | | | |
| 3 | — | Coconut oil | 80 | 20 | 55 |
| | | (C₈: 6% | | | |
| | | C₁₀: 6% | | | |
| | | C₁₂: 48% | | | |
| | | C₁₄: 20% | | | |
| | | C₁₈: 2% | | | |
| | | C₁₈: 8%) | | | |

C18″ corresponds here to a linoleic acid radical.

The melting point was determined by means of a Kofler hot bench.

The following test formulations were prepared, the percentages give the content of active substance in % by mass based on the composition. In the table below, only the surfactants are mentioned in each case.

### Test Formulations

15.0% sodium lauryl ether sulfate (SLES),
2.0% cocamidopropyl betaine (CAPB) and/or N-alkyl-N-acylglucamine (see table 2)
0.3% Jaguar Excel (galactomannan 2-hydroxypropyltrimethylammonium chloride ether)
2.0% Xiameter® MEM-1784 Emulsion (50%) (40.0-60% by weight dimethiconol, 40.0-60.0% by weight water, 1.0-5.0% by weight triethanolamine dodecylbenzylsulfonate)
0.2% Nipaguard® DMDMH (1,3-bis(hydroxymethyl)-5,5-dimethylimidazolidine-2,4-dione)

### 0.9% NaCl

ad 100% water
pH=5.5-5.6

The compositions were used as hair washing products and the silicone deposition on hair was measured.

### Experimental Procedure

### Preparation of the Hair Tresses

Hair tresses (blond, highly bleached European hair) are placed into ether for 30 minutes and then dried. Afterwards, they are washed with a solution which comprises 15% SLES.
Three hair tresses are provided for each system.
2 ml of the test formulation of each system are placed into a plastic bag for each individual tress and rubbed into the tress for 2 minutes.
The hair tresses are then rinsed with water for 1 minute.
The hair tresses are dried in air.
Three hair tresses which are not treated with a test formulation are likewise tested (standard without silicone, base line).
The hair tresses are cut into 1 cm lengths.

### Analysis

### Breaking Up of the Hair

(Emmett G. Gooch; Gretchen S. Kohl, Method to determine silicones on human hair by atomic absorption spectroscopy, J. Soc. Cosmet. Chem., 39, 382-329 (1988))
0.4 g of a hair tress are incubated for 3 days at 63° C. together with an enzyme solution (0.13 g papain, 2.0 g sodium sulfite, 100 ml water, pH 6.8 adjusted with HCl) in a closed 50 ml centrifuge tube made from polypropylene;
after cooling to room temperature, the addition of 1 ml of conc. HCl and 14 ml of methyl isobutyl ketone (MIBK) takes place;
shaking for 30 seconds;
determination of the silicone deposition in the MIBK phase by ICP-OES (atomic emission spectrometry);
(calibration: solutions of Xiameter MEM-1784 emulsion in MIBK (3.2 mg/l; 6.5 mg/l; 12.9 mg/l)).

Table 2 shows the results of the measurements. The results are moreover then shown graphically.

TABLE 2
Silicone deposition on hair

| | | Concen- | | Silicone |
|---|---|---|---|---|
| | | tration | | depo- |
| | | [% by weight] | | sition |
| | | (based on the | Di- | on hair |
| No. | System | composition) | methiconol | [ppm] |
| 1 | SLES/CAPB | 15 + 2 | yes | 80 |
| 2 | SLES/Glucamine | 15 + 2 | yes | 85 |
| | (Preparation example 2) | | | |
| 3 | SLES/Glucamine | 15 + 2 | yes | 270 |
| | (Preparation example 1) | | | |
| 4 | SLES/Glucamine | 15 + 2 | yes | 255 |
| | (Preparation example 3) | | | |
| 5 | SLES/CAPB/Glucamine | 15 + 1 + 1 | yes | 75 |
| | (Preparation example 2) | | | |
| 6 | SLES/CAPB/Glucamine | 15 + 1 + 1 | yes | 70 |
| | (Preparation example 1) | | | |
| 7 | SLES/CAPB/Glucamine | 15 + 1 + 1 | yes | 70 |
| | (Preparation example 3) | | | |
| 8 | SLES/CAPB | 15 + 2 | no | 35 |

Systems No. 3 and 4 exhibit the highest dimethiconol deposition on hair. In particular, the use of N-alkyl-N-acylglucamines, which have predominantly C₁₂- and C₁₄-acyl radicals, exhibit surprising results in the silicone deposition in combination with dimethiconol in compositions according to the invention.

1. A composition comprising:
at least one N-alkyl-N-acylglucamine as component A,
dimethiconol as component B,
at least one anionic surfactant as component C,
optionally at least one betaine surfactant as component D,
optionally at least one further surfactant as component E,
water as component F, and
optionally at least one further additive as component G.

2. The composition as claimed in claim 1, where the at least one N-alkyl-N-acylglucamine is an N-alkyl-N-acylglucamine of the formula (I), 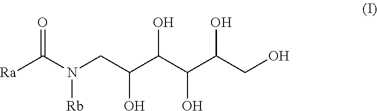
where, in the formula (I),
RₐCO is a linear or branched, saturated or unsaturated C₆-C₂₂-alkyl radical and
R_{b} is a C₁-C₄ alkyl radical.

3. The composition as claimed in claim 2, where R_{b} is a methyl radical.

4. The composition as claimed in claim 2, where the radical RₐCO is derived from myristic acid, lauric acid, palmitic acid, stearic acid, oleic acid, linoleic acid or linolenic acid.

5. The composition as claimed in claim 2, where the radical RₐCO is derived from coconut oil.

6. The composition as claimed in claim 2, where RₐCO is a C₁₂-C₁₄-acyl radical.

7. The composition as claimed in claim 6, where the at least one N-alkyl-N-acylglucamine is a mixture of
at least one N-alkyl-N-acylglucamine of the formula (I), where RₐCO is a C₁₂-acyl radical, and
at least one N-alkyl-N-acylglucamine of the formula (I), where RₐCO is a C₁₄-acyl radical.

8. The composition as claimed in claim 2, where the at least one N-alkyl-N-acylglucamine comprises more than 70% by weight, based on the N-alkyl-N-acylglucamine, of a mixture of at least one N-alkyl-N-acylglucamine of the formula (I), where RₐCO is a C₁₂-acyl radical, and at least one N-alkyl-N-acylglucamine of the formula (I), where RₐCO is a C₁₄-alkyl radical.

9. The composition as claimed in claim 2, where the at least one N-alkyl-N-acylglucamine comprises more than 90% by weight, based on the N-alkyl-N-acylglucamine, of a mixture of at least one N-alkyl-N-acylglucamine of the formula (I), where RₐCO is a C₁₂-acyl radical, and at least one N-alkyl-N-acylglucamine of the formula (I), where RₐCO is a C₁₄-acyl radical.

10. The composition as claimed in claim 1, where the composition comprises an alkyl sulfate and/or an alkyl ether sulfate as anionic surfactant.

11. The composition as claimed in claim 1, where the composition comprises a linear C₈-C₂₀-alkyl sulfate and/or a linear C₈-C₂₀-alkyl ether sulfate as anionic surfactant.

12. The composition as claimed in claim 11, where the composition comprises lauryl sulfate and/or lauryl ether sulfate as anionic surfactant.

13. The composition as claimed in claim 12, where the composition comprises sodium lauryl ether sulfate as anionic surfactant.

14. The composition as claimed in claim 1, comprising:
0.1-5.0% by weight, based on the composition, of component A,
0.1-5.0% by weight, based on the composition, of component B,
5.0-20.0% by weight, based on the composition, of component C,
0-10.0% by weight, based on the composition, of component D.

15. A hair washing product for greasy hair, dry hair, stressed hair, flaky hair, a color shampoo, baby shampoo or sport shampoo comprising the composition as claimed in claim 1.

16. The composition as claimed in claim 1, with the proviso that the composition comprises no amphoteric surfactant.

17. The composition as claimed in claim 1, where the at least one additive G is selected from the group consisting of preservatives, fragrances, dyes, surfactants, cationic polymers, thickeners and gelling agents, pigments, antimicrobial and biogenic active ingredients, moisturizing agents, stabilizers, acids and alkalies.

18. A method of caring for hair, where the hair is brought into contact with a composition comprising:
at least one N-alkyl-N-acylglucamine as component A,
dimethiconol as component B,
at least one anionic surfactant as component C,
optionally at least one betaine surfactant as component D,
optionally at least one further surfactant as component E,
water as component F, and
optionally at least one further additive as component G.

19. The method claimed in claim 17 for improving silicone deposition on the hair.

20. A method for producing a composition comprising:
at least one N-alkyl-N-acylglucamine as component A,
dimethiconol as component B,
at least one anionic surfactant as component C,
optionally at least one betaine surfactant as component D,
optionally at least one further surfactant as component E,
water as component F, and
optionally at least one further additive as component G, where the components A, B, C, F and optionally D, E, G are brought into contact with one another.
